(19) United States
(12) Patent Application Publication
(10) Pub. No.: US 2015/0073055 A1
(43) Pub. Date: Mar. 12, 2015

(54) USE OF MODAFINIL IN THE TREATMENT OF COCAINE ADDICTS

(71) Applicant: DEBREGEAS ET ASSOCIES PHARMA, Paris (FR)

(72) Inventors: Pascal Suplie, Montaure (FR); Philippe Vivet, Paris (FR)

(73) Assignee: DEBREGEAS ET ASSOCIES PHARMA, Paris (FR)

(21) Appl. No.: 14/381,896

(22) PCT Filed: Feb. 25, 2013
(86) PCT No.: PCT/FR2013/000052

§ 371 Date: Aug. 28, 2014

(30) Foreign Application Priority Data
Feb. 28, 2012 (FR) 12/00581

(51) Int. Cl. A61K 31/165; A61K 9/48; A61K 9/20

(52) U.S. Cl. CPC A61K 31/165; A61K 9/2027; A61K 9/4825
USPC 514/618

(57) ABSTRACT

The present invention relates to the application of modafinil in cocaine addiction. The modafinil used is its dextro-rotatory enantiomer (S modafinil), having a release time of less than 1 hour and wakening effect of less than 4 hours. It is absorbed orally as a pharmaceutical composition, each unit dose including from 25 to 200 mg of S modafinil. The pharmaceutical composition can be used as a substitute treatment for cocaine addicts.

Modafinil is 2-[(diphenylmethyl)sulfinyl]acetamide, the molecular formula of which is C₁₅H¹⁵NO₂S and the structural formula is:

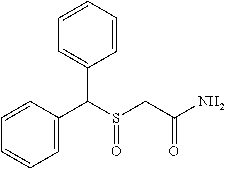 [SMILES: NC(=O)CS(=O)C(c1ccccc1)c1ccccc1]

Presently, the use of cocaine is the cause of many medical disorders and psychiatric complications such as addiction. Thus, addiction to cocaine is a serious public health problem which is only increasing. In the USA, it is reported that more than 200,000 persons in 2002 had to receive emergency care as a result of medical or psychiatric problems consecutive to the use of cocaine. Today, many molecules may be used in the treatment of addiction to cocaine. Indeed, the mode of action of cocaine at the brain level involves at least three neuromediators: GABA (Gamma-AminoButyric Acid), glutamate, and DA (dopamine). Thus, different active molecules on these mediators have been subject to an efficiency study on humans. These molecules are baclofene, topiramate, modafinil, disulfiram [B H Herman, A. Elkashef, F. Vocci—Medications for the treatment of cocaine addiction: Emerging candidates. Drug Discovery Today: Therapeutic Strategies, Vol.2, No.1, 2005], [P. Tahsili-Fahadan, G V Carr, G C Harris, and G. Aston-Jones—Modafinil blocks reinstatement of extinguished opiate-seeking in rats: mediation by a glutamate mechanism, Neuropsychopharmacology, 25, 2010, 2203-2210], [J. M. Martinez-Raga; C. Knecht and S. Cepeda—Modafinil: a useful medication for cocaine addiction? Review of the evidence from neuropharmacological, experimental and clinical studies—Current Drug Abuse Reviews, 2008, 1, 213-221].

Modafinil is a wakening drug which has been used in Europe since the nineties: it increases the wakening and diurnal alertness levels and is prescribed in the treatment of narcolepsy.

The mode of action is not entirely known but it occurs on adrenergic and glutamatergic transmissions. It binds to dopamine carriers and reduces its recapture.

It also causes inhibition of the recapture of noradrenaline at the ventrolateral optic nucleus responsible for inducing sleep.

Modafinil is marketed under the names of Modiodal, Provigil and Alertec. The administered dose varies from a taking of 100 mg to two takings of 200 mg per day; the elimination half-life is of about 14 hours in humans.

Modafinil is marketed in its racemic form which has a chiral center which in fact is a sulfur atom. However there exist two optically active isomers: the dextro-rotatory enantiomer (S) and the levo-rotatory form (R), both of these forms being a priori present in an equal amount in the racemic form.

Both enantiomers have the same pharmacological activity in animals; however, in humans, the enantiomer R has a half-life from 10 to 14 hours, The enantiomer S, as for it, has a half-life from 3 to 4 hours [Bibliographic ref.: Wong et al., J. Clin. Pharmacol., 39:30-40 (1999) ; Wong et al., J. Clin. Pharmacol., 39:281-288 (1999); Robertson et al., Clin. Pharmacokinet., 42:123-137 (2003)].

After administration, the enantiomer R would have a greater AUC (area under the curve) than the racemic form and less variability of plasma levels.

Modafinil is used in the treatment of excessive diurnal somnolence associated with narcolepsy with or without catalepsy. Excessive diurnal somnolence is characterized by difficulty in remaining awake and an increase in falling asleep periods occurring at untimely moments. The initial recommended dose is 200 mg daily administered in a single taking in the morning, or in two takings in the morning and at noon depending on the response of the patient. The doses may be increased up to 600 mg for patients having an insufficient response.

The problem of the commercial forms presently available lies in the persistence of the effect well beyond the period desired by the patient. This persistence of alertness is finally of a nature perturbing the normal sleep cycle of the patient and even induces insomnia.

Modafinil was also successfully used for children in the treatment of ADHD (attention deficit hyperactivity disorder).

In all these pathologies, strong variability is observed as regards clinical signs and it therefore proves to be necessary to proceed with individual therapeutic adjustment.

An object of the present invention is to make available to the patient, a novel oral medicinal form of S modafinil having increased bioavailability as compared with the racemic form and a shortened duration of action.

One of the goals is also to provide a formulation capable of meeting the strong interindividual variability and to therefore make available to the patient a homothetic formulation allowing easy adjustment of the administered dose.

Another object of the invention is to make available to a patient a therapeutic preparation allowing a very fast therapeutic effect as compared with the racemic form and comparatively with the S enantiomer administered alone.

Different documents of the prior art describe the use of modafinil in the treatment of cocaine addiction; thus, patent application US2001/0034373 describes the use of a daily effective dose of less than 100 mg of modafinil, and more particularly from 1 to 75 mg. The text does not bring any teaching on the formulation data or on the pharmacokinetic data.

PCT international application WO99/25329 mentions the use of benzhydryl sulfonyl derivatives, including modafinil, for reducing the somnolence effect induced by treatments with opiates. The document does not either describe the pharmaceutical compositions or any pharmacokinetic effects more specifically.

Patent application US2004/06532 mentions the use of a pharmaceutical form including between 250 and 450 mg of modafinil per therapeutic taking unit. The present invention, as for it, relates to pharmaceutical forms of S modafinil dosed with 100 mg of active ingredient, or even less.

Patent application US2009/0123545 relates an association of modafinil compounds wherein the S enantiomer is in a concentration of more than 50%. These compositions are used for increasing the vigilance, alertness condition, and more globally for increasing the stimulation of the CNS (central nervous system). The mentioned pharmaceutical data teach that the S modafinil composition is greater than 80% and that the half-life is less than 4 hours. The present invention for its part relates to specifically formulated preparations so as to obtain fast action, of less than or equal to 1 hour, and a short duration of activity, a half-life of less than 2 hours.

The text WO2007/01362 itself also describes compositions with 100 mg of S modafinil but teaches us that the induced effect persists for up to 4 hours.

The benefit of the present invention is to have a very fast action while being transient.

Thus, the present invention relates to oral therapeutic forms of S modafinil appearing as tablets, sachets or else in the form of gelatin capsules dosed with between 25 and 200 mg of active ingredient and preferentially from 50 to 100 mg.

The formulations presented here are homothetic and therefore identical regardless of the dosages administered to the patient, which contributes to reducing the strong observed variability.

Modafinil appears as a crystalline white powder practically insoluble in water and partly soluble in methanol and acetone. The result of this is low bioavailability of modafinil; it is estimated to be about 40%. Indeed, as the solubility of the active ingredient is too low, the absolute bioavailability was not able to be determined.

The compositions described here were specifically developed so as to obtain in vitro very fast dissolution and in every case greater than the one obtained with a marketed form; a discriminating dissolution method was therefore specifically developed and allowed selection of the excipients and of the manufacturing methods.

A method for a novel formulation being the object of the present invention, uses the technology of supercritical CO₂ based on the solvent power of CO₂ which may be modulated at will according to the pressure and temperature conditions which are applied to it. In the supercritical state (more than 74 bars and 31° C.), CO₂ has very particular properties. The obtained fluid is characterized by great diffusivity (of the order of that of gases), which gives it good diffusion capability, and a high density which provides it with significant transport and extraction capability.

A supercritical fluid has another advantage over other solvents: its solubility changes depending on whether its temperature or its pressure is varied. Thus it is possible to ensure that it is a solvent for certain substances at a given instant, and no longer for one at the following instant. This facilitates recovery of the substance which was dissolved.

It has appeared that modafinil has acceptable solubility in CO2.

The following step consists of spraying the dissolved active ingredient on an inert support, and then of studying the grain size of the obtained particles as well as the crystalline form of the same particles.

Different tests were carried out, first of all by using anhydrous lactose as an inert support.

Different parameters were then modified:
changing the inert support (anhydrous lactose replaced with mannitol),
increasing the loading level up to 30% of active ingredient load,
using S modafinil,
a supercritical solvent other than CO₂ .

The tests are summarized in the table below by using S modafinil and mannitol:

| | Amount of | | Amount of | | |
|---|---|---|---|---|---|
| | extracted | | collected | Collection | Theoretical |
| Test | modafinil (g) | Solubility (g/g) | formulation (g) | yield (%) | load level (%) |
| | Formulations prepared with supercritical CO₂ | | | | |
| Formulation 1 | 10.0 | 1.0.E−04 | 29.54 | 96 | 32.65 |
| Formulation 2 | 9.5 | 9.3.E−05 | 29.40 | 98 | 31.52 |
| Formulation 3 | 9.7 | 9.9.E−05 | 29.62 | 98 | 32.18 |
| | Formulations prepared with another solvent | | | | |
| Formulation 4 | 11.4 | 2.5.E−04 | 28.81 | 86 | 31.38 |
| Formulation 5 | 11.5 | 1.9.E−04 | 26.68 | 80 | 31.66 |
| Formulation 6 | 11.5 | 2.7.E−04 | 30.36 | 91 | 34.27 |

The preparations obtained previously were formulated so as to obtain tablets dosed with 2 mg of S modafinil, these tablets being intended to be administered to the rat during a pharmacokinetic study.

The method used is the following:
after weighing each of the components, the excipients are successively introduced in an increasing weight order into a mixer
the mixture is then sifted in order to remove possible clusters
the obtained mixture is then compacted and then calibrated on a grid with an aperture of 1.25 mm, at 250 rpm
compression on a tableting machine SVIAC is then carried out.

### FORMULA MADE

### DASC00512

| Raw materials | g | % |
|---|---|---|
| Sample 4 | 5.000 | 55.64 |
| Aerosil 200 | 1.250 | 13.91 |
| PVP XL | 0.358 | 3.99 |
| PVP K30 | 0.765 | 8.51 |
| Pearlitol 400 DC | 1.523 | 16.95 |
| Magnesium stearate | 0.090 | 1.00 |
| TOTAL | 8.987 | 100.00 |

The dissolution results appear in the following figure where Modiodal, Formulation 4 (powder) and compound DASC 00512 (tablets dissolved in baskets) are compared.

Another type of formulation, i.e. semi-solid gelatin capsules, was also made and tested.

### Formulation 1

| | | Amount per | |
|---|---|---|---|
| Batch: DASC00176-2 | Manufacturing | gelatin capsule | |
| Components | formula (g) | (mg) | Balance in % |
| ASC (2 × milled) = | 20.00 | 4.00 | 28.6 |
| enantiomer S | | | |
| Lipoxol 400 MED | 25.00 | 5.00 | 35.7 |
| Polysorbate 80 | 25.00 | 5.00 | 35.7 |
| TOTAL | / | 14.00 | 100.0 |

### Formulation 2

| | | Amount per | |
|---|---|---|---|
| Batch: DASC00177-2 | Manufacturing | gelatin capsule | |
| Components | formula (g) | (mg) | Balance in % |
| ASC (2 × milled) = | 20.00 | 4.00 | 28.6 |
| S enantiomer | | | |
| DUB GPE 810 | 25.00 | 5.00 | 35.7 |
| Polysorbate 80 | 25.00 | 5.00 | 35.7 |
| TOTAL | / | 14.00 | 100.0 |

### Formulation 3

| | | Amount per | |
|---|---|---|---|
| Batch: DASC00174-2 | Manufacturing | gelatin capsule | |
| Components | formula (g) | (mg) | Balance in % |
| ASC (2 × milled) = | 20.00 | 4.00 | 28.6 |
| S enantiomer | | | |
| Lipoxol 400 MED | 16.67 | 3.33 | 23.8 |
| DUB GPE 810 | 16.67 | 3.33 | 23.8 |
| Polysorbate 80 | 16.67 | 3.33 | 23.8 |
| TOTAL | / | 13.99 | 100.0 |

### Formulation 4

| | | Amount per | |
|---|---|---|---|
| Batch: DASB00175-2 | Manufacturing | gelatin capsule | Balance |
| Components | formula (g) | (mg) | in % |
| ASB (2 × milled) = racemic | 20.00 | 4.00 | 28.6 |
| Lipoxol 400 MED | 16.67 | 3.33 | 23.8 |
| DUB GPE 810 | 16.67 | 3.33 | 23.8 |
| Polysorbate 80 | 16.67 | 3.33 | 23.8 |
| TOTAL | / | 13.99 | 100.0 |

As an example, the method for manufacturing the formulation 1 will now be given, it being understood that the other formulations follow the same method, i.e. weighing the raw materials and double milling of the active ingredients (like for tablets), dispersion of the obtained powder in the excipients and distribution as gelatin capsules.
1—In a 100 ml beaker, incorporate 25 g of Lipoxol 400 MED (polyethylene glycol 400)
2—Stir at 250 rpm.
3—Incorporate at room temperature 25 g of Polysorbate 80 and stir for 2 minutes.
4—Slowly incorporate 20 g of milled active ingredient.
5—Stir for 10 minutes at 350 rpm after dispersion.
   A homogeneous and slightly viscous whitish dispersion is obtained at room temperature, titrated at 285.7 mg/g of active ingredient.

This dispersion will be incorporated into the semi-solid gelatin capsule of the HIBAR type and maintained with stirring at 200 rpm by means of stirrer provided with a marine propeller, in order to carry out the filling of the gelatin capsules.

The temperature of the hopper and of the injector will be adjusted to 27° C. It will then be proceeded with the closing of the gelatin capsules and with their stabilization.

The following figure allows comparison of the dissolution profile of the references and of the formulations.

It emerges from this figure that the four gelatin capsule formulations have a faster dissolution profile than the reference ASB dosed with 100 mg.

All the gelatin capsule formulations have greater dissolution than that of the internal references.

Among the formulations, the one based on ASB has slower dissolution than the three ASC formulations for which the dissolution profiles are identical.

It is observed, by comparing the obtained dissolution profiles, that the use of the S enantiomer of ASB and of the specific formulation shown here gives the possibility of obtaining very fast in vitro dissolution results around 90% within 30 minutes as compared with the reference products and the racemic mixture in an identical formulation.

Thus, whether this is the formulation of S modafinil obtained by the method with the supercritical fluid, or else the semi-solid gelatin capsules, the same result is achieved as regards the pharmacokinetic profile, i.e, extremely fast release, of less than 1 hour, and more specifically of the order of 30 minutes, and a transient effect, i.e., the disappearance of the persisting effect of modafinil as regards increased alertness in the 4 hours following the absorption of the pharmaceutical composition by the patient.

This double feature in terms of very fast release and transient effect is particularly of interest in the treatment of cocaine addicts.

As this has already been mentioned, cocaine addiction has become a major public health plague worldwide for which presently there are no efficient basic treatments. The patients indeed benefit from repeated and costly detoxifications in a hospital environment, at the end of which they almost inevitably relapse.

The treatment moreover is limited to non-specific psychotropic drugs during detoxification and various psychotherapies with non-proven effectiveness are carried out between these detoxification periods.

Many treatments have been tested with limited success and modafinil is the one which appears to be the most promising to international experts.

Indeed, the specific target of cocaine is the dopaminergic system of the regions of the brain involved in addiction phenomena. Cocaine inhibits neuronal membrane carriers of dopamine (DA) and thus amplifies dopaminergic reactions.

Modafinil is a non-amphetamic psychostimulating drug, for which the mechanism of action is incompletely known but has a major dopaminergic component, specifically at the DATs. Its other noradrenergic and glutamatergic effects are also linked to non-dopaminergic actions of cocaine.

These neuropharmacological similarities have lead to considering modafinil like a potential substitutive treatment of cocaine addiction.

Modafinil was tested during the years 2000 in several clinical trials with cocaine addiction patients. These tests, conducted in the United States, had a limited number of patients and gave encouraging but dis-associated results, the effectiveness being found again on certain but not all clinical and biological evaluation criteria.

The interpretation of these positive but still insufficient results resorts to the concept of imperfect substitution. Modafinil mimicks the pharmacological effect of cocaine, but its pharmacokinetic profile is too different from that of cocaine for achieving a sufficiently <<dissuasive>> substitution for the (craving) sensation and cocaine consumption in the addictive subject.

The goal is to obtain a beginning of very fast action within 15 to 30 minutes allowing the craving window to be reduced and to allow the subject to resist to addictive compulsion. The obtained product achieves a much more performing substitution than that of the native molecule.

The pharmaceutical form according to the invention results in a pharmacokinetic profile which allows this double requirement to be met: a very fast onset of action and a limited wakening effect of S modafinil, of less than 4 hours and preferentially less than 2 hours, in order to avoid the risk of insomnia linked to the prolonged wakening action of R modafinil. Indeed, covering the evening period, one of the sensitive instants for cravings and relapses, requires the product to be taken at the end of the afternoon. Therefore, there will not be any inconvenience in that the patient takes a dose of composition according to the invention at this time of the day. Said pharmaceutical form therefore is actually a therapeutic substitution form for cocaine addicts; it will appear in non-aqueous form, i.e. in solid or semi-solid form, each unit dose will contain from 25 to 200 mg of S modafinil, and preferentially from 50 to 100 mg.

Preferentially, the pharmaceutical form according to the invention will appear in an oral form, for example tablets or gelatin capsules, thereby avoiding the health risks related to administrations via injections.

1. An application of modafinil in the substitution treatment of cocaine addicts, consisting of using a pharmaceutical composition in which said modafinil is in the form of its dextro-rotary enantiomer, S modafinil, wherein the dose of S modafinil absorbed by the patient is from 50 to 100 mg per unit dose.

2. The application according to claim 1, wherein said S modafinil has a release of less than 1 hour from its absorption by the patient.

3. The application according to claim 2, wherein said release is located between 15 and 30 minutes.

4. The application according to claim 1, wherein the effect of said S modafinil is less than 4 hours from its absorption by the patient.

5. The application according to claim 4, wherein said effect is less than 2 hours.

6. The application according to claim 1, wherein said pharmaceutical composition is absorbed via an oral route.

7. The application according to claim 6, wherein said S modafinil is obtained by the technology of the supercritical fluid, said S modafinil being absorbed at the surface of granules made in an inert support.

8. The application according to claim 1, wherein said pharmaceutical composition appears as tablets.

9. The application according to claim 1, wherein said pharmaceutical composition appears as semi-solid gelatin capsules, each gelatin capsule comprising:
modafinil, in the form of its enantiomer S,
an oleic complex comprising at least one compound selected from polyethylene glycols having a molecular weight comprised between 300 and 500 daltons and from glycerides, and
an emulsifier.

10. The application according to claim 9, wherein the S modafinil/oleic complex ratio is located between 35 and 65%.

11. The application according to claim 10, wherein said ratio is located between 40 and 60%.
